(19) United States
(12) Patent Application Publication
(10) Pub. No.: US 2008/0132725 A1
(43) Pub. Date: Jun. 5, 2008

(54) Method for Producing Bis-[(3-Dimethylamino)Propyl]Amine (Dipropylene Triamine, Dpta)

(75) Inventors: Johann-Peter Melder, Bohl-Iggelheim (DE); Thomas Krug, Worms (DE)

Correspondence Address: CONNOLLY BOVE LODGE & HUTZ LLP, 1875 EYE STREET, N.W., SUITE 1100, WASHINGTON, DC, 20036, US

(73) Assignee: BASF Aktiengesellschaft, Ludwigshafen (DE)

(21) Appl. No.: 11/815,179

(22) PCT Filed: Feb. 1, 2006
(86) PCT No.: PCT/EP06/50592

§ 371 Date: Jul. 31, 2007

(30) Foreign Application Priority Data
Feb. 1, 2005 (DE) 10 2005 004854.4

(51) Int. Cl. C07C 209/00

(52) U.S. Cl. 564/469

(57) ABSTRACT

Process for preparing bis(3-aminopropyl)amine (dipropylenetriamine, DPTA) by continuous reaction of 1,3-propylenediamine (1,3-PDA) in the presence of a heterogeneous catalyst, wherein the reaction is carried out in a reaction column.

The present invention relates to a process for preparing bis(3-aminopropyl)amine (dipropylenetriamine, DPTA) by continuous reaction of 1,3-propylenediamine (1,3-PDA) in the presence of a heterogeneous catalyst.

DPTA, which has the following structural formula, is used as intermediate and hardener for epoxy resins and for the synthesis of vulcanization accelerators, emulsifiers and corrosion inhibitors.

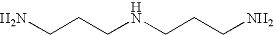

The requisite reactant 1,3-propylenediamine [H₂N—CH₂—CH₂—CH₂—NH₂; 1,3-PDA] can be prepared by known methods: for example, by reductive amination of 1,3-propanediol or 1-amino-3-propanol or by hydrogenation of malonitrile.

Symmetrical secondary amines can be prepared by catalytic amination of appropriate alcohols, aldehydes or ketones by means of corresponding primary amines with liberation of one molar equivalent of water.

Processes for preparing symmetrical secondary amines from primary amines by dimerization of the primary amine in the presence of H₂ with formation of NH₃ according to 2R—NH₂+H₂→R—NH—R+NH₃ are also known.

The dimerization of primary amines, especially of primary linear diamines, e.g. ethylenediamine (EDA) or 1,3-propylenediamine (1,3-PDA), over transition metal catalysts to form corresponding symmetrical secondary amines suffers from a multiplicity of subsequent products and secondary reactions. These include cyclic products, unwanted specifically in the diamines sector, but also higher linear products. It is generally carried out over metallic amination catalysts (e.g. Ni, Co, Cu) at elevated temperature and under superatmospheric pressure. Examples include the dimerization (conversion) of ethylenediamine (EDA) to diethylenetriamine (DETA) and the dimerization of 3-(N,N-dimethylamino)propylamine (DMAPA) to bis[(3-dimethylamino)propyl]amine (bisDMAPA).

EP-A1-1 431 273 (BASF AG) relates to a process for preparing a symmetrical secondary amine by reaction of a primary amine in the presence of hydrogen and a catalyst in whose preparation catalytically active components have been precipitated onto monoclinic, tetragonal or cubic zirconium dioxide.

EP-A1-1 270 543 (BASF AG) describes a process for preparing particular secondary amines from primary amines in the presence of hydrogen and a catalyst comprising at least one element or a compound of an element of groups VIII and IB of the Periodic Table.

DE-A1-32 48 326 (BASF AG) concerns a process for preparing polyamines from 2-cyanoethylamines over a cobalt catalyst.

German patent application 10359811.1 of Dec. 19, 2003 (BASF AG) concerns a method of increasing space/time yield (STY) in a process for preparing a symmetric secondary amine by reacting a primary amine in the presence of hydrogen and a catalyst at a temperature in the range from 50 to 250° C. under an absolute pressure in the range from 5 to 350 bar, by lowering the absolute pressure while maintaining the temperature.

Owing to the formation of ammonia in the conversion of 1,3-PDA into DPTA (2 1,3-PDA→DPTA+NH₃), the backreaction of DPTA with ammonia to form 1,3-PDA becomes increasingly significant at relatively high conversion.

Processes for the addition of alcohols onto olefins to form corresponding ethers [e.g. MTBE (methyl tert-butyl ether) and TAME (tert-amyl methyl ether)] which are carried out in a reaction column are known in the literature. The processes, which are also referred to as reactive distillation, are comprehensively described in, for example, the textbook “Reactive Distillation”, edited by K. Sundmacher and A. Kienle, Wiley-VCH publishers (2003).

Reactive distillation is also employed in the fields of esterifications, saponifications and transesterifications, preparation and saponification of acetals, preparation of alkoxides, aldol condensations, alkylations, hydrolysis of epoxides, hydration of olefins, isomerizations and hydrogenations.

The German patent applications No. 10336003.4 of Aug. 1, 2003 and No. 102004030645.1 of Jun. 24, 2004 (both BASF AG) relate to processes for preparing ethylene amines by continuous reaction of ethylenediamine (EDA) in the presence of a heterogeneous catalyst, with the reaction being carried out in a reaction column. The ethylene amines prepared are, in particular, diethylenetriamine (DETA), piperazine (PIP) and/or triethylenetetramine (TETA).

It was an object of the present invention to discover an improved economical process for the selective preparation of DPTA in high yield and space-time yield (STY).

[Space-time yields are reported in “amount of product/(catalyst volume·time)” (kg/(I_{cat.}·h)) and/or “amount of product/(reactor volume·time)” (kg/(I_{reactor}·h)].

We have accordingly found a process for preparing bis(3-aminopropyl)amine (dipropylenetriamine, DPTA) by continuous reaction of 1,3-propylenediamine (1,3-PDA) in the presence of a heterogeneous catalyst, wherein the reaction is carried out in a reaction column.

The reaction in the process of the invention proceeds according to the following equation:

2 1,3-PDA→DPTA+NH₃

According to the invention, it has been recognized that disadvantages of the processes of the prior art are avoided when the synthesis of DPTA is carried out by continuous reaction of 1,3-PDA in a reaction column (reactive distillation). As a result of DPTA being taken off continuously from the column at a point below the reaction zone (above the bottom and/or above an optional side offtake), subsequent reactions can be largely suppressed and operation with high conversion and even complete conversion of 1,3-PDA is therefore made possible.

As a result of the continuous removal of ammonia from the column (preferably at the top of the column, including as a mixture with components having boiling points lower than that of DPTA), the backreaction of DPTA to form 1,3-PDA is largely suppressed and the formation of DPTA is thus accelerated. The reaction can therefore be carried out at pressures different from, advantageously lower than, the pressure range which is optimal when using a standard fixed-bed reactor (tube reactor with fixed bed of catalyst).

The reaction column preferably has a region in which the conversion of 1,3-PDA into DPTA takes place (reaction zone), an enrichment section above the reaction zone and a stripping section below the reaction zone.

The absolute pressure in the column is preferably in the range from >0 to 20 bar, e.g. in the range from 1 to 20 bar, in particular from 5 to 10 bar.

The temperature in the region of the column in which the conversion of 1,3-PDA into DPTA takes place (reaction zone) is preferably in the range from 100 to 200° C., in particular from 140 to 160° C.

The total number of theoretical plates in the column is preferably in the range from 5 to 100, particularly preferably from 10 to 20.

The number of theoretical plates in the reaction zone is preferably in the range from 1 to 30, in particular from 1 to 20, particularly preferably from 1 to 10, e.g. from 5 to 10.

The number of theoretical plates in the enrichment section above the reaction zone is preferably in the range from 0 to 30, particularly preferably from 1 to 30, more particularly preferably from 1 to 15, in particular from 1 to 5.

The number of theoretical plates in the stripping section below the reaction zone is preferably in the range from 0 to 40, particularly preferably from 5 to 30, in particular from 10 to 20.

The 1,3-PDA can be introduced into the column in liquid or gaseous form below the reaction zone.

The 1,3-PDA can also be introduced into the column in liquid form above the reaction zone.

In the process of the invention, preference is given to feeding pure 1,3-PDA, e.g. DMAPA having a purity of >98% by weight, in particular >99% by weight, into the column.

It is also possible to use the crude 1,3-PDA product obtained after partial or complete removal of ammonia and hydrogen from the product of a reductive amination of 1,3-propanediol or 1-amino-3-propanol.

The reaction is preferably carried out in the presence of hydrogen, in particular in the presence of from 0.0001 to 1% by weight, preferably from 0.001 to 0.01% by weight, of hydrogen, in each case based on the amount of 1,3-PDA fed in.

Hydrogen is preferably introduced into the column below the reaction zone.

A mixture of ammonia, other components having a boiling point lower than that of DPTA (at the same pressure) (low boilers) and possibly hydrogen is preferably taken off at the top of the column.

The mixture taken off at the top of the column can further comprise partial amounts of unreacted 1,3-PDA.

The mixture taken off at the top can also be partially condensed and ammonia and any hydrogen can be taken off (separated off) predominantly in gaseous form and the liquefied fraction can be returned to the column as runback.

The weight ratio of the amount of runback introduced into the column to the amount of feed introduced into the column is preferably in the range from 0.1 to 30, particularly preferably from 0.5 to 10, in particular from 0.5 to 2.

Preference is given to taking off a mixture of DPTA and other components having a boiling point higher than that of DPTA (at the same pressure) (high boilers), e.g. tripropylenetriamine (TPTA), tetrapropylenepentamine (TPPA) and, if appropriate, further, higher propylene amines, which may be linear or branched, at the bottom of the column.

The mixture taken off at the bottom of the column can further comprise partial amounts of unreacted 1,3-PDA or the total amount of unreacted 1,3-PDA.

In a particular embodiment of the process, the column is divided by means of a side offtake below the reaction zone.

Preference is given to taking off unreacted 1,3-PDA via the side offtake.

The product taken off via the side offtake can further comprise DPTA.

The product obtained via the side offtake is taken off in liquid form or gaseous form.

The catalyst used in the reaction zone is preferably a catalyst comprising Ni, Co, Cu, Ru, Re, Rh, Pd and/or Pt or a shape-selective zeolite catalyst or a phosphate catalyst.

The metal or metals of the transition metal catalyst, preferably Ru, Re, Rh, Pd and/or Pt, have preferably been applied to an oxidic support material (e.g. Al₂O₃, TiO₂, ZrO₂, SiO₂) or to a zeolite or activated carbon as support material.

In a preferred embodiment, the catalyst used in the reaction zone is a catalyst comprising Pd and zirconium dioxide as support material.

The total metal content of the supported transition metal catalysts is preferably in the range from >0 to 80% by weight, particularly preferably from 0.1 to 70% by weight, more particularly preferably from 5 to 60% by weight, more particularly preferably from 10 to 50% by weight, in each case based on the weight of the support material.

In the case of the preferred supported noble metal catalysts, the total noble metal content is, in particular, in the range from >0 to 20% by weight, particularly preferably from 0.1 to 10% by weight, very particularly preferably from 0.2 to 5% by weight, more particularly preferably from 0.3 to 2% by weight, in each case based on the weight of the support material.

The heterogeneous catalysts can be accommodated in the form of fixed beds of catalysts within the column or in separate vessels outside the column. They can also be used as beds, e.g. as bed in a distillation packing, be shaped to produce packing elements or shaped bodies, for example pressed to form Raschig rings, introduced into a filter cloth and shaped to produce rolls (known as bales) or column packings, be applied to distillation packings (coating) or be used as a suspension in the column, preferably as a suspension on column trays.

In processes using heterogeneously catalyzed reactive distillations, the “bales” technology developed by CDTech can be advantageously employed.

Further technologies are specific tray constructions with packed or suspended catalysts.

Multichannel packings or cross-channel packings (cf., for example, WO-A-03/047747) allow simple introduction and discharge of catalysts which are present in particulate form (e.g. spheres, extrudates, pellets) with little mechanical stress on the catalyst.

An important point in reactive distillation is the provision of the residence time necessary for the reaction to occur. The residence time of the liquid in the column has to be increased deliberately over that in a nonreactive distillation. Use is made of special constructions of column internals, for example tray columns with bubble cap trays having a greatly increased fill level, high residence times in the outflow shafts of tray columns and/or separate external residence vessels. Backup packings offer the opportunity of increasing the residence time of the liquid by a factor of about 3 compared to columns comprising random or ordered packing.

The design of the reaction column (e.g. number of theoretical plates in the column sections, viz. enrichment section, stripping section and reaction zone, reflux ratio, etc.) can be undertaken by those skilled in the art using methods with which they are familiar.

Reaction columns are described in the literature, for example in:
“Reactive distillation of nonideal multicomponent mixtures”, U. Hoffmann, K. Sundmacher, March 1994, Trondheim/Norway,
“Prozesse der Reaktivdestillation”, J. Stichlmair, T. Frey, Chem. Ing. Tech. 70 (1998) 12, pages 1507-1516,
“Thermodynamische Grundlagen der Reaktivdestillation”, T. Frey, J. Stichlmair, Chem. Ing. Tech. 70 (1998) 11, pages 1373-1381,
WO-A-97/16243 of May 9, 1997,
DD patent 100701 of Oct. 5, 1973,
U.S. Pat. No. 4,267,396 of May 12, 1981,
“Reaktionen in Destillationskolonnen”, G. Kaibel, H.-H. Mayer, B. Seid, Chem. Ing. Tech. 50 (1978) 8, pages 586-592, and references cited therein,
DE-C2-27 14 590 of Aug. 16, 1984,
EP-B-40724 of May 25, 1983,
EP-B-40723 of Jul. 6, 1983,
DE-C1-37 01 268 of Apr. 14, 1988,
DE-C1-34 13 212 of Sep. 12, 1985,
“Production of potassium tert-butoxide by azeotropic reaction destillation”, Wang Huachun, Petrochem. Eng. 26 (1997) 11,
“Design aspects for reactive distillation”, J. Fair, Chem. Eng. 10 (1998), pages 158-162,
EP-B1-461 855 of Aug. 9, 1995,
“Consider reactive distillation”, J. DeGarmo, V. Parulekar, V. Pinjala, Chem. Eng. Prog. 3 (1992),
EP-B1-402 019 of Jun. 28, 1995,
“La distillation réaktive”, P. Mikitenko, Pétrole et Techniques 329 (1986), pages 34-38,
“Geometry and efficiency of reactive distillation bale packing”, H. Subawalla, J. González, A. Seibert, J. Fair, Ind. Eng. Chem. Res. 36 (1997), pages 3821-3832,
“La distillation réactive”, D. Cieutat, Pétrole et Techniques 350 (1989),
“Preparation of tert-amyl alcohol in a reactive distillation column”, J. González, H. Subawalla, J. Fair, Ind. Eng. Chem. Res. 36 (1997), pages 3845-3853,
“More uses for catalytic distillation”, G. Podrebarac, G. Rempel, Chem. Tech. 5 (1997), pages 37-45,
“Advances in process technology through catalytic distillation”, G. Gildert, K. Rock, T. McGuirk, CDTech, pages 103-113,
WO-A1-03/047747 of Jun. 12, 2003 (BASF AG) and
WO-A1-97/35834.

In a preferred embodiment, the process of the invention is carried out as described in WO-A1-03/047747 in a column for carrying out reactive distillations in the presence of a heterogeneous particulate catalyst, having a packing or packing elements which form intermediate spaces in the interior of the column, with the column having first and second subregions which are arranged alternately and differ in the specific surface area of the packing or packing elements so that the ratio of the hydraulic diameter for the gas stream through the packing or packing elements to the equivalent diameter of the catalyst particles is in the range from 2 to 20, preferably in the range from 5 to 10, in the first subregions, with the catalyst particles being introduced, distributed and discharged loose under the action of gravity into/in/from the intermediate spaces, and the ratio of the hydraulic diameter for the gas stream through the packing or the packing elements to the equivalent diameter of the catalyst particles is less than 1 in the second subregions and no catalyst particles are introduced into the second subregions. The column is preferably operated in terms of its gas and/or liquid throughput so that the throughput is not more than 50-95%, preferably 70-80%, of the throughput at operation under flooded conditions, cf. loc. cit., claims 9 and 10.

The work-up of the product streams obtained in the process of the invention, which comprise mostly the desired DPTA but also possibly TPTA and possibly higher polyamines and possibly unreacted 1,3-PDA, can be carried out by distillation processes known to those skilled in the art (cf., for example, PEP Report No. 138, “Alkyl Amines”, SRI International, 03/1981, pages 81-99, 117).

The distillation columns required for the purification by distillation of the desired product DPTA can be designed by those skilled in the art using methods with which they are familiar (e.g. number of theoretical plates, reflux ratio, etc.).

The mode of operation with a side offtake in the stripping section below the reaction zone of the reaction column offers particular advantages in the further work-up to obtain the DPTA in pure form.

The side offtake stream, which comprises predominantly unreacted 1,3-PDA comprises only small amounts of DPTA and high boilers.

Partial amounts or the total amount of the side stream can also be recirculated to the reaction column itself. It is particularly advantageous for the side stream to comprise predominantly 1,3-PDA and little or no DPTA.

In this mode of operation, the stream taken off at the bottom of the reaction column comprises a smaller amount of low boilers (1,3-PDA), so that the column for separating off the low-boiling components from DPTA and high boilers has to cope with a lower loading.

If the reactive distillation is carried out at low pressures, for example from 1 to 3 bar, it is also possible to obtain a bottom offtake stream which is free of 1,3-PDA at temperatures at the bottom of from about 200 to 240° C. The bottom offtake stream can be passed directly to the distillation to produce pure DPTA.

The process of the invention makes it possible to prepare DPTA with a selectivity of >70%, in particular >75%, very particularly preferably >80%, in each case based on 1,3-PDA reacted, at a 1,3-PDA conversion of >30%, in particular >40%, very particularly preferably >50%.

### EXAMPLES

### Example A

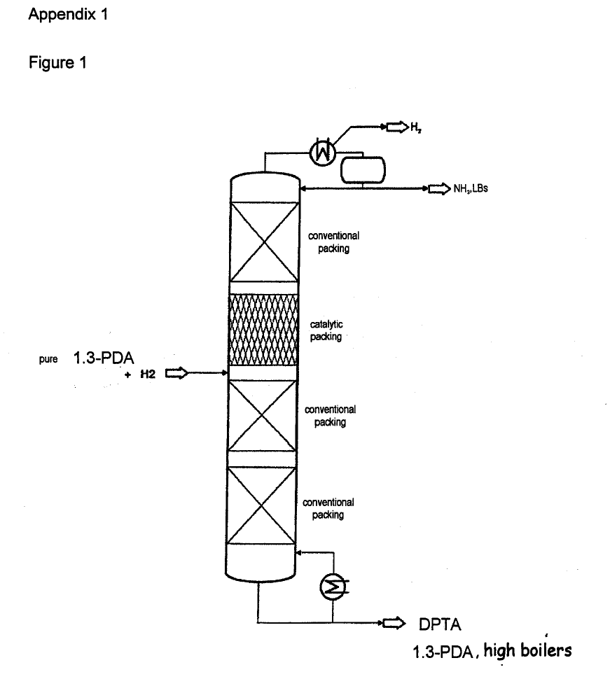

FIG. 1 in Appendix 1 shows an embodiment of the process of the invention in which pure 1,3-PDA is fed continuously together with hydrogen into the reaction column at a point below the catalytic packing and a mixture comprising DPTA, unreacted 1,3-PDA and high boilers (HBs, i.e. components having a boiling point higher than that of DPTA, e.g. TPTA, TPPA) is obtained at the bottom. Ammonia, hydrogen and low boilers (LBs, i.e. components having a boiling point lower than that of DPTA) are separated off at the top.

### Example B

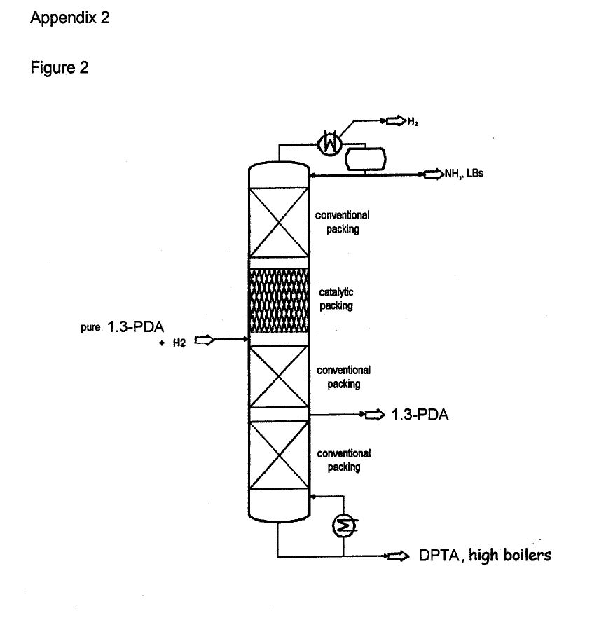

FIG. 2 in Appendix 2 shows an embodiment of the process of the invention in which pure 1,3-PDA is fed continuously together with hydrogen into the reaction column at a point below the catalytic packing and a mixture comprising DPTA and high boilers (HBs, i.e. components having a boiling point higher than that of DPTA, e.g. TPTA, TPPA) is obtained at the bottom. Ammonia, hydrogen and low boilers (LBs, i.e. components having a boiling point lower than that of DPTA) are separated off at the top.

1,3-PDA is separated off at a side offtake in the stripping section below the reaction zone of the reaction column.

1. A process for preparing bis(3-aminopropyl)amine (dipropylenetriamine, DPTA), the process comprising:
continuously reacting 1,3-propylenediamine (1,3-PDA) in the presence of a heterogeneous catalyst, wherein the reaction is carried out in a reaction column by reactive distillation.

2. The process according to claim 1, wherein the reaction column has a plurality of theoretical plates.

3. The process according to claim 1, wherein the reaction column has a region in which the conversion of 1,3-PDA into DPTA takes place (reaction zone), an enrichment section above the reaction zone and a stripping section below the reaction zone.

4. The process according to claim 1, wherein the absolute pressure in the column is in the range from >0 to 20 bar.

5. The process according to claim 1, wherein the temperature in the reaction zone is in the range from 100 to 200° C.

6. The process according to claim 1, wherein the total number of theoretical plates in the column is in the range from 5 to 100.

7. The process according to claim 1, wherein the number of theoretical plates in the reaction zone is in the range from 1 to 30.

8. The process according to claim 1, wherein the number of theoretical plates in the enrichment section above the reaction zone is in the range from 0 to 30.

9. The process according to claim 1, wherein the number of theoretical plates in the stripping section below the reaction zone is in the range from 0 to 40.

10. The process according to claim 1, wherein the catalyst used in the reaction zone is a catalyst comprising Ni, Co, Cu, Ru, Re, Rh, Pd and/or Pt or a shape-selective zeolite catalyst or a phosphate catalyst.

11. The process according to claim 1, wherein the catalyst used in the reaction zone is a catalyst comprising Pd and zirconium dioxide as support material.

12. The process according to claim 1, wherein the catalyst is installed as a bed in the reaction column.

13. The process according to claim 1, wherein the catalyst is installed as a bed in a distillation packing.

14. The process according to claim 1, wherein the catalyst is present as a coating on a distillation packing.

15. The process according to claim 1, wherein the catalyst is present in a residence vessel located outside the column.

16. The process according to claim 1, wherein the 1,3-PDA is introduced into the column in liquid form below the reaction zone.

17. The process according to claim 1, wherein the 1,3-PDA is introduced into the column in gaseous form below the reaction zone.

18. The process according to claim 1, wherein the 1,3-PDA is introduced into the column in liquid form above the reaction zone.

19. The process according to claim 1, wherein the 1,3-PDA is fed into the column in a purity of >98% by weight.

20. The process according to claim 1, wherein the reaction is carried out in the presence of hydrogen.

21. The process according to claim 20, wherein the reaction is carried out in the presence of from 0.0001 to 1% by weight of hydrogen based on the amount of 1,3-PDA fed in.

22. The process according to claim 20, wherein the hydrogen is introduced into the column below the reaction zone.

23. The process according to claim 1, wherein a mixture of ammonia, other components having a boiling point lower than that of DPTA (low boilers) and possibly hydrogen is taken off at the top of the column.

24. The process according to claim 23, wherein the mixture taken off at the top of the column further comprises partial amounts of unreacted 1,3-PDA.

25. The process according to claim 23, wherein the mixture taken off at the top is partially condensed and ammonia and any hydrogen are predominantly taken off in gaseous form and the liquefied fraction is returned to the column as runback.

26. The process according to claim 1, wherein the weight ratio of the amount of runback introduced into the column to the amount of feed introduced into the column is in the range from 0.1 to 30.

27. The process according to claim 1, wherein a mixture of DPTA and other components having a boiling point higher than that of DPTA (high boilers) is taken off from the bottom of the column.

28. The process according to claim 27, wherein the mixture taken off at the bottom of the column further comprises partial amounts of unreacted 1,3-PDA or the total amount of unreacted 1,3-PDA.

29. The process according to claim 1, wherein the column is divided by means of a side offtake below the reaction zone.

30. The process according to claim 29, wherein unreacted 1,3-PDA is taken off via the side offtake.

31. The process according to claim 29, wherein product taken off via the side offtake comprises DPTA.

32. The process according to claim 29, wherein product obtained via the side offtake is taken off in liquid form.

33. The process according to claim 29, wherein product obtained via the side offtake is taken off in gaseous form.

34. The process according to claim 1 for preparing DPTA with a selectivity of >70%, based on 1,3-PDA, at a 1,3-PDA conversion of >30%.
